(19) United States
(12) Patent Application Publication
(10) Pub. No.: US 2010/0317014 A1
(43) Pub. Date: Dec. 16, 2010

(54) COMPOSITIONS AND METHODS FOR THE ISOLATION OF NUCLEIC ACID

(75) Inventors: Mark W. Eshoo, Solana Beach, CA (US); Christopher D. Crowder, Carlsbad, CA (US); John Picuri, San Diego, CA (US)

Correspondence Address: Casimir Jones, S.C., 2275 Deming Way, Suite 310, Madison, WI, 53562, US

(73) Assignee: IBIS BIOSCIENCES, INC., Carlsbad, CA (US)

(21) Appl. No.: 12/815,946

(22) Filed: Jun. 15, 2010

Related U.S. Application Data
(60) Provisional application No. 61/186,991, filed on Jun. 15, 2009.

(51) Int. Cl. C12Q 1/68; C07H 21/00; C12N 7/00; C12N 1/20; C12P 19/34

(52) U.S. Cl. 435/6; 536/23.1; 435/235.1; 435/252.1; 435/91.2

(57) ABSTRACT

The present invention provides compositions and methods for the isolation of nucleic acids from a sample or subject. In particular, the present invention provides isolation, purification, and analysis of total DNA and RNA from a subject or sample. The compositions and methods find particular use in the isolation of nucleic acids associated with arthropods (e.g., ticks), including nucleic acid from pathogens carried by arthropods.

### CROSS-REFERENCE TO RELATED APPLICATIONS

The present application claims priority to PCT Patent Application No. ______ filed Jun. 15, 2010 (filed concurrently herewith) and U.S. Provisional Application Ser. No. 61/186,991 filed Jun. 15, 2009, the entirety of each of which is herein incorporated by reference.

### FIELD OF THE INVENTION

The present invention provides compositions and methods for the isolation of nucleic acids from a sample or subject. In particular, the present invention provides isolation, purification, and analysis of total DNA and RNA from a subject or sample. The compositions and methods find particular use in the isolation of nucleic acids associated with arthropods (e.g., ticks), including nucleic acid from pathogens carried by arthropods.

### BACKGROUND

Ticks harbor numerous bacterial, protozoal, and viral pathogens that can cause serious infections in humans and domestic animals. Active surveillance of the tick vector can provide insight into the frequency and distribution of important pathogens in the environment. Nucleic-acid based detection of bacterial and viral pathogens requires the extraction of both DNA and RNA from ticks, as many viral pathogens harbored by ticks are RNA viruses. Traditional methods for nucleic acid extraction are limited to extraction of either DNA or the RNA from a sample.

Vector-borne diseases caused over 148,000 deaths and more than 12.5 million disability-adjusted life years (DALYs) worldwide in 2002 (Beaglehole et al. 2004. World Health Report 2004: changing history. World Health Organization., herein incorporated by reference in its entirety). The tick vector can transmit a variety of pathogens ranging from viruses and bacteria to protozoa (de la Fuente et al. 2008. Front Biosci 13: 6938-46., herein incorporated by reference in its entirety). The diseases caused by bacterial pathogens range from life threatening infections, such as tularemia and Rocky Mountain spotted fever virus, to potentially chronic infections, like Lyme disease (de la Fuente et al. 2008. Front Biosci 13: 6938-46., herein incorporated by reference in its entirety). Manifestations of protozoan infections such as Babesiosis can range from flu-like symptoms to severe recurring infections and death in humans (Vannier et al. 2008. Infect Dis Clin North Am 22: 469-88, viii-ix., herein incorporated by reference in its entirety) and can also affect livestock and pets (Bock et al. 2004. Parasitology 129 Suppl: S247-69., herein incorporated by reference in its entirety).

Tick-borne RNA viruses from a wide range of families can also cause serious illness and death. In North America, the Powassan virus and Deer Tick fever virus, both Flaviviruses, have been found in four species of Ixodes, as well as Dermacentor andersoni ticks (Romero & Simonsen. 2008. Infect Dis Clin North Am 22: 545-59, x., herein incorporated by reference in its entirety). Colorado tick fever virus, a Coltivirus from the Reoviridae family, is transmitted by D. andersoni and infects 200-400 people annually. In the United States alone, there were over 23,000 reported cases of tick-transmitted diseases in 2006 (McNabb & Jajosky. 2008. Summary of Notifiable Diseases—United States, 2006, Morbidity and Mortality Weekly Report. Centers for Disease Control and Prevention, herein incorporated by reference in its entirety). In Europe and Asia, tick-borne encephalitis (TBE), another Flavivirus, affects more than 10,000 people each year (Lindquist & Vapalahti. 2008. Lancet 371: 1861-71., herein incorporated by reference in its entirety). Additionally, the Crimean-Congo hemorrhagic fever virus, an RNA virus that belongs to the Bunyaviridae family, can be transmitted by ticks of the Hyalommai genus; cases have been found throughout Europe, Africa, and Asia (Ergonul. 2006. Lancet Infect Dis 6: 203-14., herein incorporated by reference in its entirety).

Most direct pathogen detection assays employ PCR and require the efficient homogenization of the ticks, lysis of the pathogens, and extraction of the nucleic acids from inhibitors of PCR. A number of methods have been reported for extracting nucleic acids from ticks, such as crushing frozen ticks with a mortar and pestle, homogenizing the ticks with small beads in a bead-beater, or cutting apart the tick with a scalpel (Halos et al. 2004. Vet Res 35: 709-13., Hill & Gutierrez. 2003. Med Vet Entomol 17: 224-7., Exner & Lewinski. 2003. Diagn Microbiol Infect Dis 46: 235-40, Moriarity et al. 2005. J Med Entomol 42: 1063-7., herein incorporated by reference in their entireties). However, none of these methods can be used to effectively and simultaneously extract both DNA and RNA from a single tick. Extraction of both DNA and RNA is vital for pathogen surveillance, as some tick-borne pathogens are RNA viruses (Romero & Simonsen. 2008. Infect Dis Clin North Am 22: 545-59, x., herein incorporated by reference in its entirety) and co-infections are common (Clay et al. 2008. Mol. Ecol., herein incorporated by reference in its entirety).

### SUMMARY

The present invention provides compositions and methods for the isolation of nucleic acids from a sample or subject. In particular, the present invention provides isolation, purification, and analysis of total DNA and RNA from a subject or sample. The compositions and methods find particular use in the isolation of nucleic acids associated with arthropods (e.g., ticks), including nucleic acid from pathogens carried by arthropods.

In some embodiments, the present invention provides a method for isolation of DNA and/or RNA from a sample comprising agitating a sample with zirconia/yttria beads or other solid surface materials. In some embodiments, the present invention comprises the steps of: (a) providing: (i) a sample, wherein the sample comprises one or more organisms, (ii) zirconia/yttria beads, and (iii) liquid reagent, (b) combining the sample, liquid reagent, and beads into a mixture, (c) agitating the mixture, wherein agitating results in the breakdown of the sample and the release of the DNA and RNA into the liquid, (d) extracting the liquid from the beads, and (e) purifying the DNA and RNA from contaminants in the liquid. In some embodiments, the one or more organisms comprises an arthropod. In some embodiments, the arthropod comprises an arachnid, Leptotrombidium sp., Liponyssiodes sanguineus, Dermacentor, xodid ticks, Ornithodoros sp., Ixodes sp., Dermacentor variabilis, Amyblyomma americanum, Crustaceans, Copepod, Cyclops sp., Crabs, crayfish, insects, lice, Pediculus humanus, Xenopsylla cheopis, rodent fleas, Triatoma, Panstrongylus sps., Beetles, flour beetle, Fly, gnat, Glossina sp., Simulium sp., Chrysops sp., Phlebotomus sp., Lutzomyia sp., Phlebotomus sp., mango flies, mosquito, Aedes aegypti, Culiseta melanura, Coquillettidia pertubans, Aedes vexans, Aedes triseriatus, Culex sp., or Culex tarsalis. In some embodiments, the arthropod comprises a tick. In some embodiments, the one or more organisms comprise one or more pathogenic organisms. In some embodiments, the

one or more pathogenic organisms comprise Rickettsia tsutsugamushi, Rickettsia akari, Francisella tularensis, Rickettsia rickettsia, Borrelia sp., Babesia microti, Borrelia burgdorferi, Ehrlichia canis, E. sennetsu, E. chaffeensis, E. equi, E. phagocytophilia, CTF virus, Eyach virus, Russian Spring-Summer Encephalitis, Louping Ill Encephalitis, Langat virus, Powassan virus, Omsk hemorrhagic fever virus, Nairobi sheep disease virus, Crimean-Congo hemorrhagic fever virus, Diphyllobothrium latum, Diphyllobothrium spirometra, Dracunculus medinensis, Paragonimus westermani, Rickettsia prowazekii, Bartonella Quintana, Borrelia recurrentis, Yersina pestis, Rickettsia typhi, Hymenolepsis diminuta, Diphylidium caninum, Trypanosoma cruzi, Hymenolepsis nana, Trypanosoma brucei rhodesiense, T.b. gambiense, Onchocerca volvulus, Francisella tularensis, Leishmania donovani, L. tropica, L. braziliensis, Bartonella bacilliformis, Loa boa, Sandfly fever Naples virus, Sandfly fever Sicilian virus, Rift valley fever virus, Plasmodium falciparum, Plasmodium malariae, Plasmodium vivax, Plasmodium ovale, Wuchereria bancrofti, Brugia malayi, Dirofilaria immitis, Yellow fever virus, Dengue fever virus, Eastern Equine Encephalitis virus, La Crosse Encephalitis virus, St. Louis Encephalitis virus, Venezualan Equine Encephalitis virus, Western Equine Enchephalitis virus, Chikungunya virus, Mayaro fever virus, Mucambo fever virus, O'Nyong-Nyong fever virus, Pixuna fever virus, Ross River fever virus, Nile fever virus, Japanese encephalitis virus, West Nile fever virus, Zika fever virus, Wesselsbron fever virus, Kyasanur forest disease virus, Oropouche virus, Bunyamwera, Bwamba fever virus, Guama fever virus, Oropouche fever virus, California Enchephalitis virus, Chandipura fever virus, or Piry fever virus. In some embodiments, the zirconia/yttria beads comprise greater than 70% ZrO₂. In some embodiments, the present invention further comprises amplifying the isolated DNA and RNA to generate an amplicon. In some embodiments, the present invention further comprises determining the mass or base composition of the amplicon. In some embodiments, the present invention further comprises identifying one or more organisms by comparing the determined mass or base composition to a database of masses or base compositions from known organisms.

The present invention further provides kits comprising zirconia/yttria beads and one or more additional components useful, necessary, or sufficient for conducting a nucleic acid isolation or analysis procedures, examples of which are described elsewhere herein. Such additional components include, but are not limited to, oligonucleotide primers or probes that are complementary to the nucleic acid, buffers (e.g., buffers compatible with mass spectroscopy analysis of nucleic acid molecules), enzymes (e.g., polymerases, ligases, etc.), and software (e.g., for data collection and analysis). For example, in some embodiments, the present invention provides a kit comprising zirconia/yttria beads and a nucleic acid primer, wherein the primer is complementary to a sequence from a tick-borne pathogen. In some embodiments, the kit comprises two nucleic acid primers. In some embodiments, the primers are complementary to a nucleic acid region conserved among multiple different tick-borne pathogens. In some embodiments, the multiple different tick-borne pathogens comprise different species of tick-borne pathogens. In some embodiments, the multiple different tick-borne pathogens comprise different genus of tick-borne pathogens. In some embodiments, the multiple different tick-borne pathogens comprise one or more tick-borne viruses. In some embodiments, the primers are complementary to a nucleic acid region flanking a variable region. In some embodiments, the variable region comprises different sequences among different tick-borne pathogens. In some embodiments, the different tick-borne pathogens comprise different species of tick-borne pathogens. In some embodiments, the different tick-borne pathogens comprise different genus of tick-borne pathogens. In some embodiments, the different tick-borne pathogens comprise one or more tick-borne viruses. In some embodiments, the kit further comprises one or more buffers compatible with mass spectrometry analysis.

The present invention further provides reaction mixtures (e.g., residing in a tube, wells, etc.) comprising zirconia/yttria beads in contact with arthropod tissue and/or nucleic acid derived therefrom (e.g., nucleic acid from a bacteria, virus, or other organism harbored by an arthropod). In some embodiments, the present invention provides a reaction mixture comprising a reaction vessel containing zirconia/yttria beads and tick-borne pathogen nucleic acid. In some embodiments, the reaction mixture further comprises one or more nucleic acid primers. In some embodiments, the reaction mixture further comprises one or more buffers compatible with PCR analysis and/or mass spectroscopy.

### DESCRIPTION OF FIGURES

The foregoing summary and detailed description may be better understood when read in conjunction with the accompanying drawings which are included by way of example and not by way of limitation.

FIG. 1 shows gels of nucleic acids isolated from a single adult Dermacentor variabilis tick. (A) Total nucleic acids, (B) DNA, and (C) RNA from the same sample. The left lane in each panel is a DNA ladder. The ribosomal RNA bands are clearly visible in gels A and C.

FIG. 2 shows detection of B. burgdorferi from a single I. scapularis nymph. (A) Gel analysis of the PCR amplification of the Borrelia flagellin gene and (B) the rplB gene. (C) Detection of the B. burgdorferi B31 flaB gene and (D) the rplB gene by PCR/ESI-MS on the T5000 biosensor.

FIG. 3 shows a histogram of a comparison of the nucleic acid extraction efficiency of zirconia/yttria beads, zirconia/silica beads, and glass beads in S. aureus.

FIG. 4 shows a histogram of a comparison of the nucleic acid extraction efficiency of zirconia/yttria beads, zirconia/silica beads, and glass beads in Candida Albicans.

FIG. 5 shows a histogram of the nucleic acid extraction efficiency of beads from Bacillus cerus spores (zirconia/yttria beads vs. zirconia/silica beads).

FIG. 6 shows a histogram of the nucleic acid extraction efficiency of beads from Bacillus cerus spores (AL buffer v. ALT buffer).

### DEFINITIONS

It is to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this invention pertains. In describing and claiming the present invention, the following terminology and grammatical variants will be used in accordance with the definitions set forth below.

As used herein, the term “about” means encompassing plus or minus 10%. For example, about 90% refers to a range encompassing between 81% and 99% nucleotides. As used herein, the term “about” is synonymous with the term approximately.

As used herein, the term “amplicon” or “bioagent identifying amplicon” refers to a nucleic acid generated using the primer pairs described herein. The amplicon is typically double stranded DNA; however, it may be RNA and/or DNA:RNA. In some embodiments, the amplicon comprises DNA complementary to herpesvirus DNA, or cDNA. In some embodiments, the amplicon comprises sequences of conserved regions/primer pairs and intervening variable region. As such, the base composition of any given amplicon may include the primer pair, the complement of the primer pair, the conserved regions and the variable region from the bioagent that was amplified to generate the amplicon. One skilled in the art understands that the incorporation of the designed primer pair sequences into an amplicon may replace the native sequences at the primer binding site, and complement thereof. In certain embodiments, after amplification of the target region using the primers the resultant amplicons having the primer sequences are used to generate the molecular mass data. Generally, the amplicon further comprises a length that is compatible with mass spectrometry analysis. Bioagent identifying amplicons generate base compositions that are preferably unique to the identity of a bioagent.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. Amplification is not limited to the strict duplication of the starting molecule. For example, the generation of multiple cDNA molecules from a limited amount of RNA in a sample using reverse transcription (RT)-PCR is a form of amplification. Furthermore, the generation of multiple RNA molecules from a single DNA molecule during the process of transcription is also a form of amplification.

As used herein, “viral nucleic acid” includes, but is not limited to, DNA, RNA, or DNA that has been obtained from a virus or a sample containing a virus. Viral DNA and RNA can either be single-stranded (of positive or negative polarity) or double-stranded.

As used herein, a “bioagent” means any biological organism or component thereof or a sample containing a biological organism or component thereof, including microorganisms or infectious substances, or any naturally occurring, bioengineered or synthesized component of any such microorganism or infectious substance or any nucleic acid derived from any such microorganism or infectious substance. Those of ordinary skill in the art will understand fully what is meant by the term bioagent given the instant disclosure. Still, a non-exhaustive list of bioagents includes: cells, cell lines, human clinical samples, mammalian blood samples, cell cultures, bacterial cells, viruses, viroids, fungi, protists, parasites, rickettsiae, protozoa, animals, mammals or humans, arthropods, insects, arachnids, ticks, etc. Samples may be alive, non-replicating, dead, in a vegetative state (for example, vegetative bacteria or spores), frozen, etc.

The term “detect”, “detecting” or “detection” refers to an act of determining the existence or presence of one or more targets (e.g., bioagent nucleic acids, amplicons, etc.) in a sample.

As used herein, the term “etiology” refers to the causes or origins, of diseases or abnormal physiological conditions.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method.

As used herein, the term “molecular mass” refers to the mass of a compound as determined using mass spectrometry, for example, ESI-MS. Herein, the compound is preferably a nucleic acid. In some embodiments, the nucleic acid is a double stranded nucleic acid (e.g., a double stranded DNA nucleic acid). In some embodiments, the nucleic acid is an amplicon. When the nucleic acid is double stranded the molecular mass is determined for both strands. In one embodiment, the strands may be separated before introduction into the mass spectrometer, or the strands may be separated by the mass spectrometer (for example, electro-spray ionization will separate the hybridized strands). The molecular mass of each strand is measured by the mass spectrometer.

As used herein, the term “nucleic acid molecule” refers to any nucleic acid containing molecule, including but not limited to, DNA or RNA. The term encompasses sequences that include any of the known base analogs of DNA and RNA including, but not limited to, 4 acetylcytosine, 8-hydroxy-N6-methyladenosine, aziridinylcytosine, pseudoisocytosine, 5-(carboxyhydroxyl-methyl)uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein a “sample” refers to anything capable of being subjected to the compositions and methods provided herein. In some embodiments, the sample comprises or is suspected to comprise one or more nucleic acids capable of analysis by the methods. Preferably, the samples comprise nucleic acids (e.g., DNA, RNA, cDNAs, etc.). In some embodiments, the samples are “mixture” samples, which comprise nucleic acids from more than one subject or individual. In some embodiments, the methods provided herein comprise purifying the sample or purifying the nucleic acid(s) from the sample. In some embodiments, the sample is purified or unpurified nucleic acid. As used herein, the term “subject” refers to any animal (e.g., arthropod, tick, mammal, human, etc.), including, but not limited to, arthropods, arachnids, insects, ticks, flies, mites, humans, non-human primates, vertebrates, pigs, rodents, and the like.

### DETAILED DESCRIPTION

Extraction of nucleic acids from ticks is challenging (Mauel et al. J. Med. Entomol. 36 (1999) 649-652., Schwartz et al. Am. J. Trop. Med. Hyg. 56 (1997) 339-342., herein incorporated by reference in their entirety). Ticks possess a hard chitinous exoskeleton that must be disrupted before extraction of nucleic acids. DNA extracted from ticks is highly susceptible to degradation (Hill and Gutierrez. Med. Vet. Entomol. 17 (2003) 224-227., Hubbard et al. Exp. Appl. Acarol. 19 (1995) 473-478., herein incorporated by reference in their entirety). The presence of polymerase inhibitors has also been implicated in both engorged and unfed ticks (Hubbard et al. Exp. Appl. Acarol. 19 (1995) 473-478., Schwartz et al. Am. J. Trop. Med. Hyg. 56 (1997) 339-342., Sparagano et al. Exp. Appl. Acarol. 23 (1999) 929-960., herein incorporated by reference in their entirety). The present invention provides compositions and methods for the efficient isolation of nucleic acids from ticks, as well as other organisms and samples. In particular, the present invention provides isolation, purification, and analysis of DNA and/or RNA from a sample using a zirconia/yttria substrate (e.g. beads). The compositions and methods find particular use in the isolation of nucleic acids associated with arthropods (e.g., ticks), including nucleic acid from pathogens carried by arthropods.

In some embodiments, the present invention provides a bead-based method (e.g. using zirconia/yttria beads) for extraction of total nucleic acid (e.g. DNA and RNA) from a subject or sample (e.g. arthropods (e.g. tick (e.g. Ixodes scapularis))) and for detection of infectious agents (e.g. bacteria (e.g. Borrelia burgdorferi), viruses, protozoa, etc.). In some embodiments, compositions and methods provided are sensitive enough to detect infectious agents from a single-arthropod sample. In some embodiments, the present methods are applicable to a variety of arthropod vectors, including fleas and mosquitoes. In some embodiments, zirconium/yttria beads are employed to homogenize samples (e.g. ticks) and physically lyse bacteria and protozoa; proteases are used to lyse viruses. In some embodiments, a silica-gel column or other purification component is then used to remove inhibitors that might interfere with subsequent nucleic acid analysis steps (e.g., PCR) and cellular debris from nucleic acids of interest. In some embodiments, methods of the present invention are automatable (e.g. automated on a QIACUBE apparatus).

In some embodiments, the present invention provides compositions and methods to simultaneously extract DNA and RNA from a sample (e.g. organism, mammal, arthropod, tick, etc.). In some embodiments, method and compositions of the present invention are configured to extract total nucleic acid from a sample or subject(s) (e.g. >0.1% of total nucleic acid, >1% of total nucleic acid, >2% of total nucleic acid, >5% of total nucleic acid, >10% of total nucleic acid, >20% of total nucleic acid, >50% of total nucleic acid, >75% of total nucleic acid, >90% of total nucleic acid, >95% of total nucleic acid, >99% of total nucleic acid, etc.). In some embodiments, a sample comprises part or all of a subject or organism. In some embodiments, a sample comprises a subject or organism and any pathogenic organisms therein or thereon. In some embodiments, a sample comprises multiple organisms. In some embodiments, a sample comprises multiple organisms in symbiosis (e.g. parasitism, mutualism, and/or commensalism). In some embodiments, a sample comprises portions of multiple subjects or organisms. In some embodiments, a sample is a primary organism (e.g. human tissue, part or all of an arthropod, etc.) and any secondary organisms (e.g. viruses, bacteria, protozoa, etc.) therein or thereon. In some embodiments, the identity of a primary or secondary organism is unknown prior to the isolation and/or analysis of the nucleic acid from the sample. In some embodiments, one or more (e.g. 1, 2, 3, 4, 5, 6, 7, 8, 9, 10 . . . 20 . . . 50 . . . 100 . . . ) secondary organisms are present on or in the primary organism. In some embodiments, a sample comprises one or more primary organisms and one or more secondary organisms. In some embodiments, a sample comprises one or more primary organisms of a single species. In some embodiments, a sample comprises more than one specie of the primary organism. In some embodiments, a sample comprises multiple types (e.g., classes, orders, families, genuses, species) of secondary organisms (e.g. bacteria, viruses, etc.). In some embodiments, samples and/or subjects are live, dead, preserved, decaying, frozen, vegetative, whole, partial, etc.

In some embodiments, a sample comprises one or more primary organisms. In some embodiments, a primary organism is a eukaryote, eubacteria, archaebacteria, virus, animal, plant, fungus, protist, invertebrate, vertebrate, mammal, non-human primate, human, rodent, canine, feline, equine, bovine, arthropod, chelicerate, pycnogonid, merostomata, arachnid, amblypygi, araneae, opilione, palpigradi, pseudoscorpionida, ricinulei, schizomida, scorpions, solifugae, thelyphonida, acarina, acariformes, parasitiform, holothyrida, mesostigmata, ixodida, ixodidae, argasidae, nuttalliellidae, etc. In some embodiments, a sample comprises only a portion of a primary organism (e.g. organ, tissue, section, etc.). In some embodiments, a sample consists of a single whole primary organism.

In some embodiments, a sample comprises one or more secondary organisms. In some embodiments a secondary subject is a eukaryote, eubacteria, archaebacteria, virus, fungi, protist, rhizaria, cercozoa, retaria, amoeboids, flagellates, amoebozoa, molds, amoeboflagellates, yeast, algae, gram-negative bacteria, gram-positive bacteria, actinobacteria, firmicutes, tenericutes, aquificae, bacteroidetes/chlorobi, chlamydiae/verrucomicrobia, deinococcus-thermus, fusobacteria, gemmatimonadetes, nitrospirae, proteobacteria, spirochaetes, synergistetes, acidobacteria, chloroflexi, chrysiogenetes, cyanobacteria, deferribacteres, dictyoglomi, fibrobacteres, planctomycetes, thermodesulfobacteria, thermotogae, dna viruses, rna viruses, adenoviruses, herpesviruses, poxviruses, parvoviruses, reoviruses, picornaviruses, togaviruses, orthomyxoviruses, rhabdoviruses, retroviruses, hepadnaviruses, etc.

In some embodiments, the present invention provides isolation of nucleic acids (e.g. DNA and RNA) from arthropod vectors including, but not limited to arachnids, Leptotrombidium sp. (red mites), Liponyssiodes sanguineus (mouse mite), Dermacentor, xodid ticks, Ornithodoros sp., Ixodes sp., Dermacentor variabilis, Amyblyomma americanum, Crustaceans, Copepod, Cyclops sp., Crabs, crayfish, insects, lice, Pediculus humanus, fleas, Xenopsylla cheopis, rodent fleas, Triatoma, Panstrongylus sps., Beetles, flour beetle, Fly, gnat, Glossina sp. (tsetse fly), Simulium sp. (black fly), Chrysops sp., Phlebotomus sp., Lutzomyia sp. (sandflies), Phlebotomus sp., mango flies, mosquito, Aedes aegypti, Culiseta melanura, Coquillettidia pertubans, Aedes vexans, Aedes triseriatus, Culex sp., Culex tarsalis, etc.

In some embodiments, nucleic acids (e.g. DNA and RNA) are from one or more pathogenic organisms including, but not limited to Rickettsia tsutsugamushi, Rickettsia akari, Francisella tularensis, Rickettsia rickettsia, Borrelia sp., Babesia microti, Borrelia burgdorferi, Ehrlichia canis, E. sennetsu, E. chaffeensis, E. equi, E. phagocytophilia, CTF virus, Eyach virus, Russian Spring-Summer Encephalitis, Louping Ill Encephalitis, Langat virus, Powassan virus, Omsk hemorrhagic fever virus, Nairobi sheep disease virus, Crimean-Congo hemorrhagic fever virus, Diphyllobothrium latum, Diphyllobothrium spirometra, Dracunculus medinensis, Paragonimus westermani, Rickettsia prowazekii, Bartonella Quintana, Borrelia recurrentis, Yersina pestis, Rickettsia typhi, Hymenolepsis diminuta, Diphylidium caninum, Trypanosoma cruzi, Hymenolepsis nana, Trypanosoma brucei rhodesiense, T.b. gambiense, Onchocerca volvulus, Francisella tularensis, Leishmania donovani, L. tropica, L. braziliensis, Bartonella bacilliformis, Loa boa, Sandfly fever Naples virus, Sandfly fever Sicilian virus, Rift valley fever virus, Plasmodium falciparum, Plasmodium malariae, Plasmodium vivax, Plasmodium ovale, Wuchereria bancrofti, Brugia malayi, Dirofilaria immitis, Yellow fever virus, Dengue fever virus, Eastern Equine Encephalitis virus, La Crosse Encephalitis virus, St. Louis Encephalitis virus, Venezualan Equine Encephalitis virus, Western Equine Enchephalitis virus, Chikungunya virus, Mayaro fever virus, Mucambo fever virus, O'Nyong-Nyong fever virus, Pixuna fever virus, Ross River fever virus, Nile fever virus, Japanese encephalitis virus, West Nile fever virus, Zika fever virus, Wesselsbron fever virus, Kyasanur forest disease virus, Oropouche virus, Bunyamwera, Bwamba fever virus, Guama fever virus, Oropouche fever virus, California Enchephalitis virus, Chandipura fever virus, or Piry fever virus.

In some embodiments, compositions and methods of the present invention provide monitoring for known and emerging vector-borne pathogens (e.g. pathogens in tick and/or other arthropods). In some embodiments, the present invention provides compositions, kits, methods, and reagents which find utility in the identification of tick-borne pathogens. Particular primers, probes and other reagents that find use in these embodiments are described in the patent application PCT/US09/45660, herein incorporated by reference in its entirety. As a number of tick viral pathogens are RNA viruses, compositions and methods provide analysis of both DNA and RNA to determine the viral pathogens, as well as bacterial and protozoan pathogens, present. The compositions and methods herein find utility in isolating and/or analyzing nucleic acids from live, ethanol-preserved, or frozen samples (e.g. ticks). In some embodiments, extraction of nucleic acids from live ticks affords the highest quality RNA.

In some embodiments, the compositions and methods described herein find utility in vector surveillance of arthropods including ticks, fleas, mosquitoes, mites, and various flies. Many arthropod vectors can transmit a combination of viruses (RNA and DNA), bacteria, and protozoa (Kalluri et al. 2007. PLoS Pathog 3: 1361-71., herein incorporated by reference in its entirety). Mosquitoes transmit RNA viruses such as West Nile and Dengue, as well as the etiological agent of malaria, the protozoan Plasmodium falciparum. Fleas and mites have been shown to transmit bacteria that cause a number of diseases and sandflies transmit the protozoa that cause Leishmaniasis and viruses that cause Sandfly fever. Other reports have described homogenization by bead-beating the vector, but only in context of DNA isolation and from multiple ticks (Moriarity et al. 2005. J Med Entomol 42: 1063-7., herein incorporated by reference in its entirety) or fleas (Allender et al. 2004. Biotechniques 37: 730, 732, 734., herein incorporated by reference in its entirety). The method described herein find utility in the detection of the above vectors as well as others known to those in the art, allowing the detection of both the RNA viruses as well as the DNA from various pathogens. In some embodiments, when the nucleic acid isolation methods of the present invention are used in combination with PCR/ESI-MS, a single vector provides sufficient nucleic acid for accurate analysis.

In some embodiments, along with the extraction of both DNA and RNA, the present compositions and methods provide isolation of nucleic acid from various types of tissues and clinical samples (e.g. tissues and clinical samples from humans, non-human primates, canines, felines, bovine, equine, rodent, etc.). Tail or skin tissues or tough organs such as heart and spleen are quickly homogenized and nucleic acids extracted without lengthy overnight digestions or manual homogenization by mortar and pestle. In some embodiments, compositions and methods of the present invention find utility in the analysis of skin biopsies.

In some embodiments, the present invention provides zirconia/yttria compositions (e.g. surfaces, beads, substrates, devices, etc.). In some embodiments, the present invention provides zirconia/yttria compositions for the purification, isolation, preparation, and/or analysis of nucleic acids (e.g. total DNA and RNA). In some embodiments, compositions of the present invention comprise yttrium and/or zirconium. In some embodiments zirconia/yttria compositions comprise ZrO₂ (zirconium dioxide) and/or Y₂O₃ (yttrium oxide). In some embodiments, the present invention provides yttria stabilized zirconia (YSZ). In some embodiments, the present invention provides zirconium-oxide based ceramic. In some embodiments, the crystal structure of zirconium oxide is made stable at room temperature by an addition of yttrium oxide. In some embodiments, compositions comprise 95% ZrO₂ and 5% Y₂O₃. In some embodiments, compositions comprise approximately 95% ZrO₂ and 5% Y₂O₃. In some embodiments, compositions comprise at least 70% ZrO₂ (e.g. 70% ZrO₂, 75% ZrO₂, 80% ZrO₂, 85% ZrO₂, 90% ZrO₂, 95% ZrO₂, 99% ZrO₂, >99% ZrO₂, etc.). In some embodiments, compositions comprise greater than about 70% ZrO₂ (e.g. approximately 71% ZrO₂, approximately 75% approximately 80% ZrO₂, approximately 85% ZrO₂, approximately 90% ZrO₂, approximately 95% ZrO₂, approximately 99% ZrO₂, >99% ZrO₂, etc.). In some embodiments, compositions comprise 30% or less Y₂O₃ (e.g. 30% Y₂O₃, 25% Y₂O₃, 20% Y₂O₃, 15% Y₂O₃, 10% Y₂O₃, 5% Y₂O₃, 1% Y₂O₃, <1% Y₂O₃, etc.). In some embodiments, compositions comprise less than about 30% Y₂O₃ (e.g. approximately 29% Y₂O₃, approximately 25% Y₂O₃, approximately 20% Y₂O₃, approximately 15% Y₂O₃, approximately 10% Y₂O₃, approximately 5% Y₂O₃, approximately 1% Y₂O₃, <1% Y₂O₃, etc.). In some embodiments, compositions of the present invention may comprise additional compounds and/or compositions in addition to zirconia/yttria. In some embodiments, compositions may comprise calcia-stabilized zirconia, magnesia-stabilized zirconia, ceria-stabilized zirconia or alumina-stabilized zirconia. In some embodiments, compositions may contain an amount of impurities acceptable to those of skill in the art. In some embodiments, compositions are devoid of silica. In some embodiments, compositions are devoid of a substantial amount of silica. In some embodiments, the present invention provides beads of a suitable size for molecular biology purposes as would be understood by one of skill in the art. In some embodiments, the present invention provides beads with a mean diameter of greater than 1 μm (e.g. about 2 μm, about 5 μm, about 10 μm, about 20 μm, about 50 μm, about 100 μm, about 200 μm, about 500 μm, about 1 mm, about 2 mm, about 5 mm, about 1 cm, >1 cm, etc.).

In some embodiments, the present invention provides buffers and reagents for use with zirconia/yttria compositions (e.g. for storage, use in purification of nucleic acid, charging, cleaning, etc.). In some embodiments, the present invention provides an appropriate salts (e.g. NaCl, KOH, MgCl₂, etc.) and salt concentration (e.g. high salt, low salt, 1 mM, 2 mM, 5 mM, 10 mM, 20 mM, 50 mM, 100 mM, 200 mM, 500 mM, 1 M, etc.) for use with zirconia/yttria compositions. In some embodiments, buffers for use with zirconia/yttria compositions may include, but are not limited to H₃PO₄/NaH₂PO₄, Glycine, Citric acid, Acetic acid, Citric acid, MES, Cacodylic acid, H₂CO₃/NaHCO₃, Citric acid, Bis-Tris, ADA, Bis-Tris Propane, PIPES, ACES, Imidazole, BES, MOPS, NaH₂PO₄/Na₂HPO₄, TES, HEPES, HEPPSO, Triethanolamine, Tricine, Tris, Glycine amide, Bicine, Glycylglycine, TAPS, Boric acid (H₃BO₃/Na₂B₄O₇), CHES, Glycine, NaHCO₃/Na₂CO₃, CAPS, Piperidine, Na₂HPO₄/Na₃PO₄, combinations thereof, etc.

In some embodiments, bead-beating with very high density yttria-stabilized zirconium-oxide beads provides very rapid results and higher quality nucleic acids than those obtain from protocols employing lengthy incubation steps when nucleases have the opportunity to degrade nucleic acids. Bead-beating is a sample homogenization and cell lysis method in which a biological sample (e.g. organism, tissue, cell) is agitated (e.g. vigorously agitated) with beads (e.g. glass or other material) to break up the sample and lyse cells through physical means. Higher density beads allow for shorter bead-beating times and result in overall higher yields than the standard and less dense zirconium-silica beads. The mechanical nature of the lysis by bead beating in conjunction with the enzymatic and chemical lysis resulted in improved effectiveness against hard to lyse organisms such as Gram-positive bacteria and spores, ensuring that the nucleic acids of all the microbiota of the tick are obtained.

Experiments performed during development of embodiments of the present invention demonstrated that the compositions and methods of the present invention provide high-quality DNA and RNA from individual ticks and from the pathogens that reside within the tick. For example, as described in the experimental examples below, nucleic acids obtained from compositions and methods of the present invention provide the identification of B. burgdorferi in ticks by both PCR and PCR/ESI-MS. Methods can be automated, for example using the Qiagen QiaCube (Qiagen, Valencia, Calif.) to increase the throughput of tick pathogen surveillance.

Provided herein are methods, compositions, kits, and related systems for the isolation, purification, and analysis of total DNA and RNA from a subject or sample. In some embodiments, analysis of DNA and/or RNA isolated and or purified by the present invention comprises amplification and/or mass spectrometry analysis of DNA and/or RNA. In some embodiments, primers are selected to hybridize to conserved sequence regions of nucleic acids derived from a subject or sample (e.g. bioagent) and which flank variable sequence regions to yield an identifying amplicon that can be amplified and that is amenable to molecular mass determination. In some embodiments, the molecular mass is converted to a base composition, which indicates the number of each nucleotide in the amplicon. Systems employing software and hardware useful in converting molecular mass data into base composition information are available from, for example, Ibis Biosciences, Inc. (Carlsbad, Calif.), for example the Ibis T5000 Biosensor System, and are described in U.S. patent application Ser. No. 10/754,415, filed Jan. 9, 2004, incorporated by reference herein in its entirety. In some embodiments, the molecular mass or corresponding base composition of one or more different amplicons is queried against a database of molecular masses or base compositions indexed to bioagents and to the primer pair used to generate the amplicon. A match of the measured base composition to a database entry base composition associates the sample bioagent to an indexed bioagent in the database. Thus, the identity of the unknown bioagent is determined. No prior knowledge of the unknown bioagent is necessary to make an identification. In some instances, the measured base composition associates with more than one database entry base composition. Thus, a second/subsequent primer pair is used to generate an amplicon, and its measured base composition is similarly compared to the database to determine its identity in triangulation identification. Furthermore, the methods and other aspects of the invention can be applied to rapid parallel multiplex analyses, the results of which can be employed in a triangulation identification strategy. Thus, in some embodiments, the present invention provides rapid throughput and does not require nucleic acid sequencing or knowledge of the linear sequences of nucleobases of the amplified target sequence for bioagent detection and identification.

Particular embodiments of the mass-spectrum based detection methods are described in the following patents, patent applications and scientific publications, all of which are herein incorporated by reference as if fully set forth herein: U.S. Pat. Nos. 7,108,974; 7,217,510; 7,226,739; 7,255,992; 7,312,036; 7,339,051; US patent publication numbers 2003/0027135; 2003/0167133; 2003/0167134; 2003/0175695; 2003/0175696; 2003/0175697; 2003/0187588; 2003/0187593; 2003/0190605; 2003/0225529; 2003/0228571; 2004/0110169; 2004/0117129; 2004/0121309; 2004/0121310; 2004/0121311; 2004/0121312; 2004/0121313; 2004/0121314; 2004/0121315; 2004/0121329; 2004/0121335; 2004/0121340; 2004/0122598; 2004/0122857; 2004/0161770; 2004/0185438; 2004/0202997; 2004/0209260; 2004/0219517; 2004/0253583; 2004/0253619; 2005/0027459; 2005/0123952; 2005/0130196 2005/0142581; 2005/0164215; 2005/0266397; 2005/0270191; 2006/0014154; 2006/0121520; 2006/0205040; 2006/0240412; 2006/0259249; 2006/0275749; 2006/0275788; 2007/0087336; 2007/0087337; 2007/0087338 2007/0087339; 2007/0087340; 2007/0087341; 2007/0184434; 2007/0218467; 2007/0218467; 2007/0218489; 2007/0224614; 2007/0238116; 2007/0243544; 2007/0248969; WO2002/070664; WO2003/001976; WO2003/100035; WO2004/009849; WO2004/052175; WO2004/053076; WO2004/053141; WO2004/053164; WO2004/060278; WO2004/093644; WO 2004/101809; WO2004/111187; WO2005/023083; WO2005/023986; WO2005/024046; WO2005/033271; WO2005/036369; WO2005/086634; WO2005/089128; WO2005/091971; WO2005/092059; WO2005/094421; WO2005/098047; WO2005/116263; WO2005/117270; WO2006/019784; WO2006/034294; WO2006/071241; WO2006/094238; WO2006/116127; WO2006/135400; WO2007/014045; WO2007/047778; WO2007/086904; WO2007/100397; WO2007/118222; Ecker et al., Ibis T5000: a universal biosensor approach for microbiology. Nat Rev Microbiol. 2008 Jun. 3.; Ecker et al., Identification of Acinetobacter species and genotyping of Acinetobacter baumannii by multilocus PCR and mass spectrometry. J Clin Microbiol. 2006 August; 44(8):2921-32.; Ecker et al., Rapid identification and strain-typing of respiratory pathogens for epidemic surveillance. Proc Natl Acad Sci USA. 2005 May 31; 102(22):8012-7. Epub 2005 May 23.; Wortmann et al., Genotypic Evolution of Acinetobacter baumannii Strains in an Outbreak Associated With War Trauma. Infect Control Hosp Epidemiol. 2008 June; 29(6):553-555.; Hannis et al., High-resolution genotyping of Campylobacter species by use of PCR and high-throughput mass spectrometry. J Clin Microbiol. 2008 April; 46(4):1220-5.; Blyn et al., Rapid detection and molecular serotyping of adenovirus by use of PCR followed by electrospray ionization mass spectrometry. J Clin Microbiol. 2008 February; 46(2):644-51.; Eshoo et al., Direct broad-range detection of alphaviruses in mosquito extracts. Virology. 2007 Nov. 25; 368(2):286-95.; Sampath et al., Global surveillance of emerging Influenza virus genotypes by mass spectrometry. PLoS ONE. 2007 May 30; 2(5):e489.; Sampath et al., Rapid identification of emerging infectious agents using PCR and electrospray ionization mass spectrometry. Ann N Y Acad. Sci. 2007 April; 1102:109-20.; Hujer et al., Analysis of antibiotic resistance genes in multidrug-resistant Acinetobacter sp. isolates from military and civilian patients treated at the Walter Reed Army Medical Center. Antimicrob Agents Chemother. 2006 December; 50(12):4114-23.; Hall et al., Base composition analysis of human mitochondrial DNA using electrospray ionization mass spectrometry: a novel tool for the identification and differentiation of humans. Anal Biochem. 2005 Sep. 1; 344(1):53-69.; Sampath et al., Rapid identification of emerging pathogens: coronavirus. Emerg Infect Dis. 2005 March; 11(3):373-9., each of which is herein incorporated by reference in its entirety.

### EXPERIMENTAL

While the present invention has been described with specificity in accordance with certain of its embodiments, the following examples serve only to illustrate the invention and are not intended to limit the same. In order that the invention disclosed herein may be more efficiently understood, examples are provided below. It should be understood that these examples are for illustrative purposes only and are not to be construed as limiting the invention in any manner.

### Example 1

### Compositions and Methods for Nucleic Acid Extraction

### Tick Lysis and Nucleic Acid Extraction.

Ticks were obtained from the Oklahoma State University tick-rearing facility (Stillwater, Okla.), California State vector control departments, and field collections in Tennessee and New York. A modification of the QIAGEN Virus MinElute kit (QIAGEN, Valencia, Calif.) was used to extract RNA and DNA from the ticks. Ticks were homogenized in 0.5-mL screw-cap tubes (Sarstedt, Newton, N.C.). The tubes were filled with 750 mg of 2.0 mm zirconia/yttria beads, 150 mg of 0.1 mm zirconia/yttria beads (Glen Mills, Clifton, N.J.), and 450 μL of lysis buffer consisting of 419 μL of QIAGEN ATL buffer, 6 μL of a 1 mg/mL stock of sonicated poly A (Sigma-Aldrich, St Louis, Mo.), and 25 μL proteinase K solution (QIAGEN). The tubes were shaken in a BioSpec Mini Bead Beater 16 (BioSpec, Bartlesville, Okla.) for 1 min for nymphal ticks or 2.5 min for adult ticks. The samples then were then centrifuged for 2 min at 6000 g in a benchtop microcentrifuge. A 400-μL aliquot of the recoverable supernatant was transferred to a fresh microcentrifuge tube and 400 μL of AL buffer was added. The tubes were briefly mixed by vortexing for 30 s, pulse centrifuged, and incubated at 37° C. for 10 min. Subsequently, 480 μL of 100% ethanol was added and samples were mixed by vortexing for 30 s and then centrifuged to remove liquid from the tube cap. The samples were then loaded onto the QIAGEN MinElute column in two parts: First, 750 μL of sample was loaded and then centrifuged at 6000 g for 1 min. The flow through was discarded and the remaining sample was loaded onto the column and the column was again centrifuged at 6000 g for 1 min. The column was transferred to a new collection tube and 500 μL of AW1 wash buffer (QIAGEN) was added and the column was centrifuged for 1 min at 6000 g. The flow through was discarded and 500 μL of AW2 wash buffer (QIAGEN) was added to the column which was again centrifuged for 1 min at 6000 g. After discarding the flow though, 500 μL of 100% ethanol was pipetted onto the column and the column was centrifuged for 1 min at 6000 g. The flow through was discarded and the column was dried by spinning at 6000 g for 1 min to remove any remaining ethanol. Nucleic acids were eluted by adding 100 μL of AVE elution buffer (QIAGEN) to the column, incubating at room temperature for 5 min, and centrifuging for 1 min at 6000 g. This method was automated on the QIACUBE (QIAGEN) following the bead-beating step and manual collection of the supernatant.

### DNase and RNase Treatments.

To examine the RNA yield using the extraction protocol, the DNA was digested with DNase I, RNase-free, enzyme (Roche, Indianapolis, Ind.). Briefly, a 17.5 μL aliquot of the nucleic acid extract was mixed with 2 μL of 10×DNase incubation buffer (Roche) and 0.5 μL of DNase I (5 units). The reaction was incubated at 37° C. for 15 min with mixing and brief centrifugation every 5 min. To examine the DNA yield of the extraction, the RNA was digested with RNase, DNase-free enzyme (Roche). Specifically, 45 μL of the sample was mixed with 1 μL of RNase (0.5 μg) and incubated for 30 min at 37° C. with mixing and brief centrifuging every 10 min. The resulting enzyme-treated extracts were visualized on a 1% agarose gel with 8 μl of the All Purpose Hi-Lo ladder (Bionexus, Oakland, Calif.) which ranges from 10,000 bp to 50 bp in size.

### PCR.

Detection of Borrelia DNA was performed using PCR primers targeting the gene encoding flagellin and the rplB gene. The forward and reverse primer sequences used for Borrelia flaB gene were 5′-TGCTGAAGAGCTTGGAATGCA-3′ (SEQ ID NO. 1) and 5′-TACAGCAATTGCTTCATCTTGATTTGC-3′ (SEQ ID NO. 2), respectively. The forward and reverse primer sequences used for the detection of the Borrelia rplB gene were 5′-TCCACAAGGTGGTGGTGAAGG-3′ (SEQ ID NO. 3) and 5′-TCGGCTGTCCCCAAGGAG-3′ (SEQ ID NO. 4), respectively. PCR was performed using 1 μl extract in a reaction mix consisting of 1 unit of Immolase Taq polymerase (Bioline USA, Taunton, Mass.), 20 mM Tris (pH 8.3), 75 mM KCl, 1.5 mM MgCl2, 0.4 M betaine, 800 μM dNTP mix (Bioline USA), 20 mM sorbitol (Sigma), 2 μg/mL sonicated poly A RNA (Sigma), 500 μg/mL of ultrapure BSA (Invitrogen, Carlsbad, Calif.) and 250 nM of each primer. The following PCR cycling conditions were used on an MJ Dyad 96-well thermocycler (Bio-Rad, Hercules, Calif.): 95° C. for 10 min, followed by 8 cycles of 95° C. for 30 s, 48° C. for 30 s, and 72° C. 30 s, with the initial 48° C. annealing temperature increasing 0.9° C. each cycle. PCR was then continued for 37 additional cycles of 95° C. for 15 s, 56° C. for 20 s, and 72° C. for 20 s. The PCR ended with a final extension of 2 min at 72° C. followed by a 4° C. hold. The PCR product was visualized on a 4% agarose gel.

### Total Nucleic Acid Quantitation.

Total nucleic acid tick extracts (2 μL) were quantified by absorbance 260/280 measurement using a Nanodrop ND-1000 (Thermo Scientific, Wilmington, Del.) using QIAGEN AVE buffer as the blank. Water extractions were also quantitated to determine the contribution of the poly A carrier to the overall yield; this value was subtracted from the samples to determine the total nucleic acid yields from tick samples.

### Mass Spectrometry and Base Composition Analysis.

Mass spectrometry was performed on an Ibis T5000 Biosensor (Ibis Biosciences, Carlsbad, Calif.). After PCR amplification, 30 μL aliquots of each PCR reaction were desalted and purified using a weak anion exchange protocol described previously (Ecker et al. 2006. J Clin Microbiol 44: 2921-32., herein incorporated by reference in its entirety). Accurate mass (±1 ppm), high-resolution (M/dM>100,000 FWHM) mass spectra were acquired for each sample using high-throughput ESI-MS protocols described previously (Sampath et al. 2007. PLoS ONE 2: e489., herein incorporated by reference in its entirety). For each sample, approximately 1.5 μL of analyte solution was consumed during the 74 s spectral acquisition. Raw mass spectra were post-calibrated with an internal mass standard and deconvolved to monoisotopic molecular masses. Unambiguous base compositions were derived from the exact mass measurements of the complementary single-stranded oligonucleotides (Muddiman et al. 1997. Anal Chem 69: 1543-9., herein incorporated by reference in its entirety).

### Example 2

### Nucleic Acid Extraction from Ticks

DNA and RNA Extracted from Single Ticks.

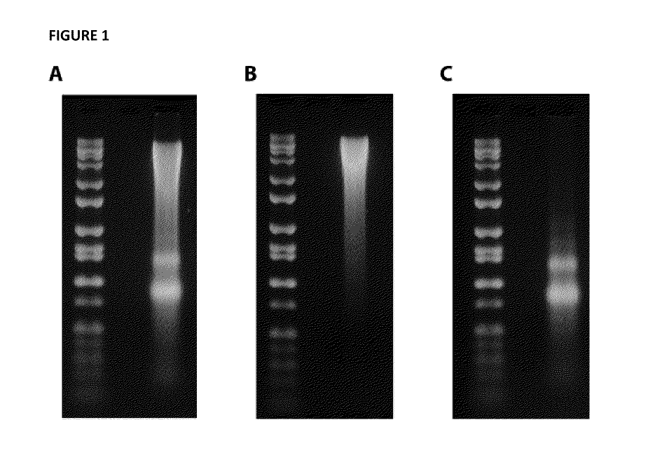

The quality of the extraction was determined by total nucleic acid yield and visual inspection of the DNA and ribosomal RNA bands in a gel. A time course of 30 s to 3 min, in 30 s increments, was used for the nymphal optimization and a range of 1 min to 6 min was used for adult tick optimization. Optimal yields were observed when nymphs and adults were bead-beaten for 1 min and 2.5 min, respectively. Increases in bead-beating times did not affect the total nucleic acid yield significantly but degradation of the ribosomal RNA bands was observed at longer times. Total nucleic acid was then isolated from a single flat adult Dermacentor varabilis tick. Gel analysis was performed of total nucleic acids, DNA, and RNA (total nucleic acids treated with RNase and DNase, respectively) from this tick (SEE FIG. 1).

The total nucleic acid yields from various tick species was determined using laboratory-reared and field-collected ticks. Table 1 shows the mean, standard deviation, and 95% confidence interval for the extraction of total nucleic acids from adult Dermacentor varabilis, Demermacentor occidentalis, Amblyomma americanum, and Ixodes scapularis ticks. To account for the nucleic acid contribution from the poly A carrier, extractions were performed without the ticks and the ratio of 260 nm to 280 nm was determined on a Nanodrop-1000. The blank extractions were found to have a mean of 4.4 μg of DNA (n=5, σ=0.18). The amount of total nucleic acid from each tick was calculated by subtracting this amount from the measured values.

TABLE 1
Total nucleic acid yields from adult ticks

| | | Total μg nucleic acid per tick | | |
|---|---|---|---|---|
| | | | | 95% |
| | | | Standard | confidence |
| Species | n | Mean | deviation | interval |
| Dermacentor varabilis | 7 | 18.4 | 3.9 | 2.9 |
| Dermacentor occidentalis | 11 | 9.0 | 2.5 | 1.0 |
| Amblyomma americanum | 4 | 8.8 | 1.6 | 2.4 |
| Ixodes scapularis | 15 | 5.8 | 2.0 | 1.0 |

Detection of a Tick-Borne Bacterial Pathogen from Single Ticks.

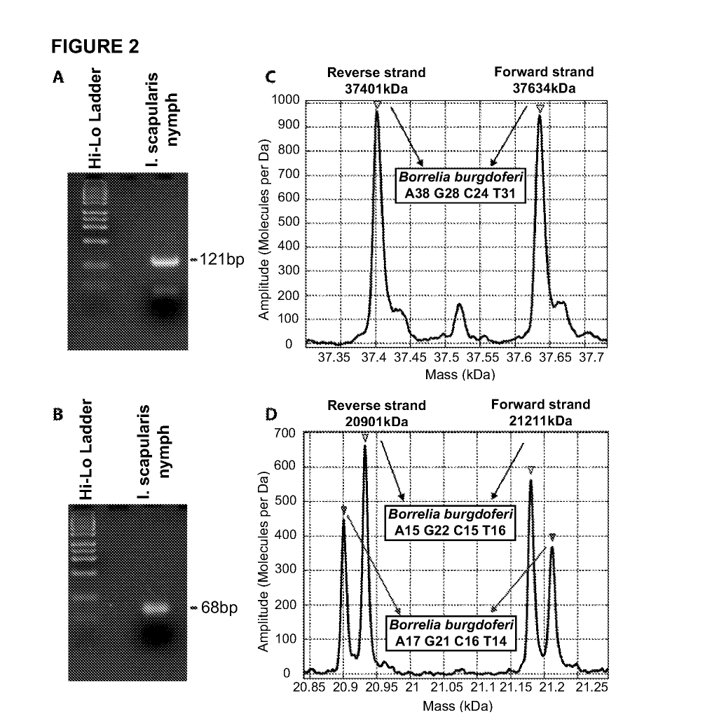

Extraction of total nucleic acids was performed on individual field-collected ticks. The extracts were then analyzed by PCR with primers targeting the Borrelia flaB and rplB genes. The genes encoding flagellin and rplB by PCR in an extract from an Ixodes scapularis nymph collected in New York were detected (SEE FIG. 2). PCR/ESI-MS nucleotide base compositions were consistent with the expected base compositions of the flaB and rplB genes of B. burgdorferi (SEE FIGS. 2C and 2D). The primers for rplB also amplified what we believe is the Rickettsial endosymbiont of I. scapularis (SEE FIG. 2D). methods of the present invention have also been used to detect B. burgdorferi and B. miyamotoi in a number of field-collected adult and nymphal I. scapularis from New York and adult Ixodes pacificus ticks from California.

### Example 3

### Nucleic Acid Extraction from Whole Blood

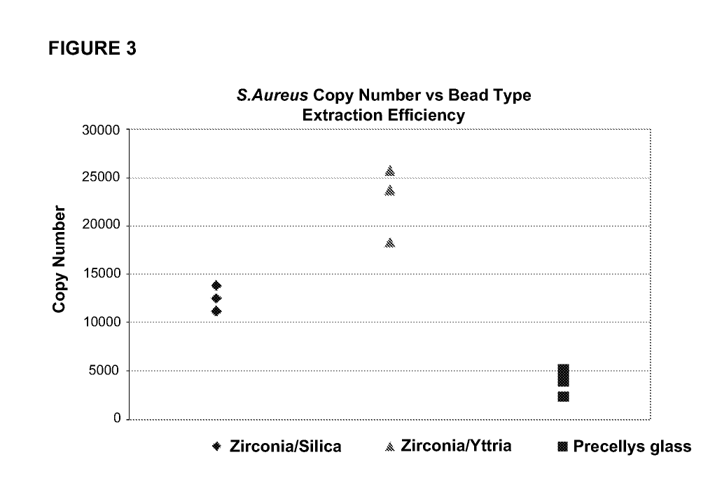

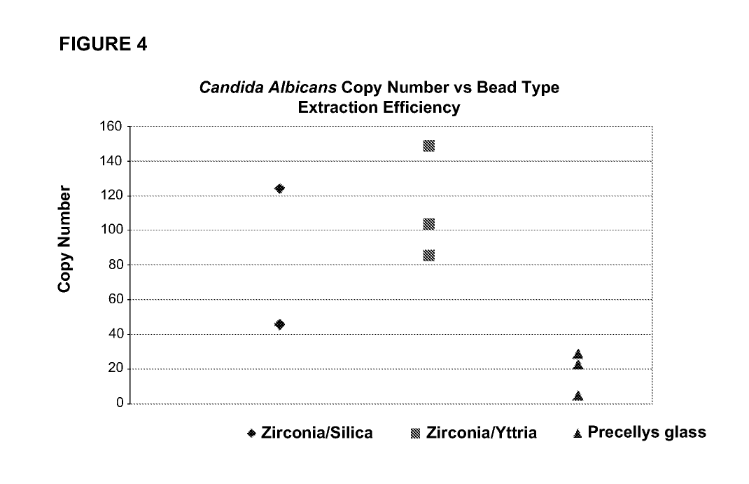

Experiments were preformed during development of embodiments of the present invention to compare the performance of zirconia/yttria beads with zirconia/silica beads and glass beads. The beads tested had an average diameter of 100 μm. Bead beating was performed using PRECELLYS23 bead beater. Extraction was performed using Kingfisher Flex/Abbott Blood Isolation Protocol. 100 CFU/ml S aureus and 100 CFU/ml Candida Albicans in whole blood were used as samples. QPCR was used to quantitate the extraction. Zirconia/yttria beads demonstrated superior performance to both the zirconia/silica beads and glass beads in the extraction of DNA and RNA from S aureus (SEE FIG. 3) and Candida Albicans (SEE FIG. 4).

### Example 4

### Nucleic Acid Extraction from Bacillus cereus Spores

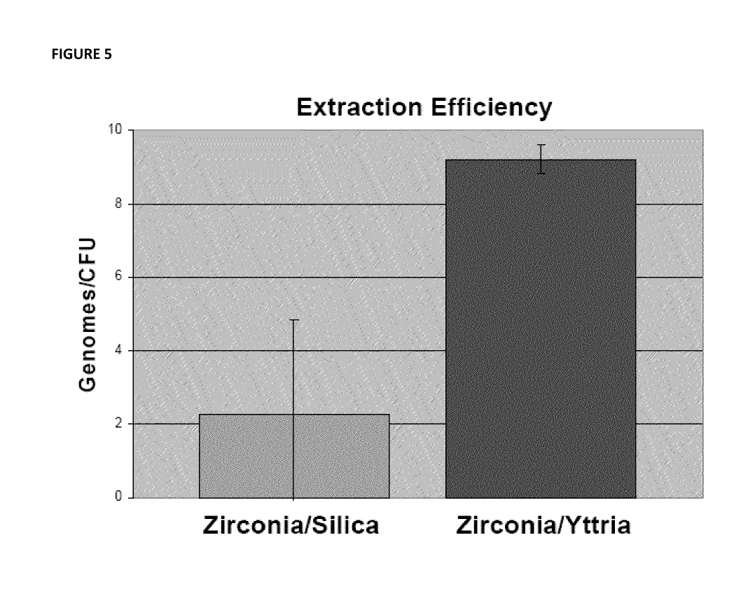

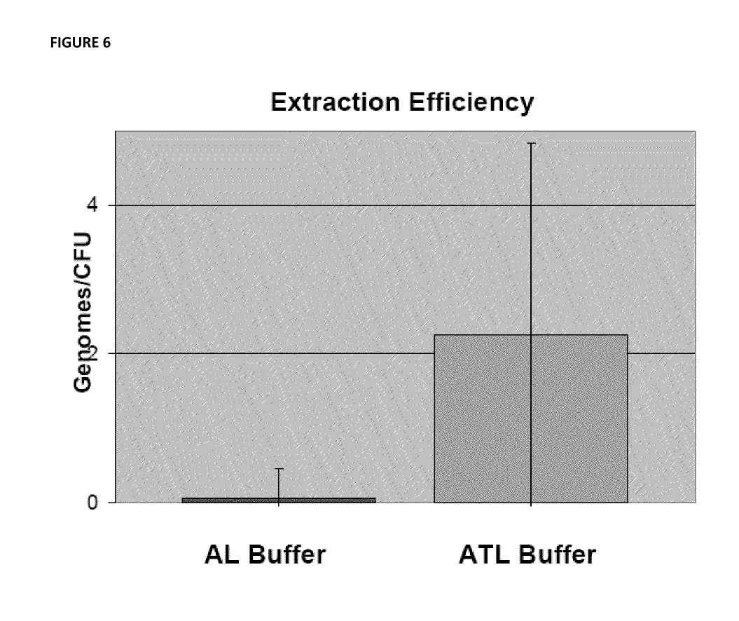

Experiments were preformed during development of embodiments of the present invention to compare the performance of zirconia/yttria beads with zirconia/silica beads at extraction of nucleic acids from Bacillus cereus spores. Bacillus cereus spores are known to those in the art to be difficult to lyse. Extractions were performed using the QIAGEN QIAMP mineulte column. Extracts were prepared with 1000 CFU of B. cereus spores. QPCR was used to quantitate the results. Bead beating was performed in Biospec bead beater for 90 seconds. Zirconia/yttria beads exhibited significantly greater extraction efficiency that zirconia/silica beads (SEE FIG. 5). ALT low salt buffer yielded significantly more efficient nucleic acid extraction efficiency than AL high salt buffer (SEE FIG. 6).

1. A method for isolation of DNA or RNA from a sample comprising agitating a sample with zirconia/yttria beads.

2. The method of claim 1, comprising isolation of DNA and RNA from said sample.

3. The method of claim 2, comprising the steps of:
a) providing:
i) a sample, wherein said sample comprises one or more organisms;
ii) zirconia/yttria beads; and
iii) liquid reagent;
b) combining said sample, said liquid reagent, and said beads into a mixture;
c) agitating said mixture, wherein agitating results in the breakdown of said sample and the release of said DNA and RNA into said liquid;
d) extracting said liquid from said beads; and
e) purifying said DNA and RNA from contaminants in said liquid.

4. The method of claim 3, wherein said one or more organisms comprises an arthropod.

5. The method of claim 4, wherein said arthropod comprises an arachnid, Leptotrombidium sp., Liponyssiodes sanguineus, Dermacentor, xodid ticks, Ornithodoros sp., Ixodes sp., Dermacentor variabilis, Amyblyomma americanum, Crustaceans, Copepod, Cyclops sp., Crabs, crayfish, insects, lice, Pediculus humanus, Xenopsylla cheopis, rodent fleas, Triatoma, Panstrongylus sps., Beetles, flour beetle, Fly, gnat, Glossina sp., Simulium sp., Chrysops sp., Phlebotomus sp., Lutzomyia sp., Phlebotomus sp., mango flies, mosquito, Aedes aegypti, Culiseta melanura, Coquillettidia pertubans, Aedes vexans, Aedes triseriatus, Culex sp., or Culex tarsalis.

6. The method of claim 4, wherein said arthropod comprises a tick.

7. The method of claim 3, wherein said one or more organisms comprise one or more pathogenic organisms.

8. The method of claim 7, wherein said one or more pathogenic organisms comprise Rickettsia tsutsugamushi, Rickettsia akari, Francisella tularensis, Rickettsia rickettsia, Borrelia sp., Babesia microti, Borrelia burgdorferi, Ehrlichia canis, E. sennetsu, E. chaffeensis, E. equi, E. phagocytophilia, CTF virus, Eyach virus, Russian Spring-Summer Encephalitis, Louping Ill Encephalitis, Langat virus, Powassan virus, Omsk hemorrhagic fever virus, Nairobi sheep disease virus, Crimean-Congo hemorrhagic fever virus, Diphyllobothrium latum, Diphyllobothrium spirometra, Dracunculus medinensis, Paragonimus westermani, Rickettsia prowazekii, Bartonella Quintana, Borrelia recurrentis, Yersina pestis, Rickettsia typhi, Hymenolepsis diminuta, Diphylidium caninum, Trypanosoma cruzi, Hymenolepsis nana, Trypanosoma brucei rhodesiense, T.b. gambiense, Onchocerca volvulus, Francisella tularensis, Leishmania donovani, L. tropica, L. braziliensis, Bartonella bacilliformis, Loa boa, Sandfly fever Naples virus, Sandfly fever Sicilian virus, Rift valley fever virus, Plasmodium falciparum, Plasmodium malariae, Plasmodium vivax, Plasmodium ovale, Wuchereria bancrofti, Brugia malayi, Dirofilaria immitis, Yellow fever virus, Dengue fever virus, Eastern Equine Encephalitis virus, La Crosse Encephalitis virus, St. Louis Encephalitis virus, Venezualan Equine Encephalitis virus, Western Equine Enchephalitis virus, Chikungunya virus, Mayaro fever virus, Mucambo fever virus, O'Nyong-Nyong fever virus, Pixuna fever virus, Ross River fever virus, Nile fever virus, Japanese encephalitis virus, West Nile fever virus, Zika fever virus, Wesselsbron fever virus, Kyasanur forest disease virus, Oropouche virus, Bunyamwera, Bwamba fever virus, Guama fever virus, Oropouche fever virus, California Enchephalitis virus, Chandipura fever virus, or Piry fever virus.

9. The method of claim 1, wherein said zirconia/yttria beads comprise greater than 70% ZrO₂.

10. The method of claim 2, further comprising amplifying said isolated DNA or RNA to generate an amplicon.

11. The method of claim 10, further comprising determining the mass or base composition of said amplicon.

12. The method of claim 11, further comprising identifying said one or more organisms by comparing the determined mass or base composition to a database of masses or base compositions from known organisms.

13. A kit comprising zirconia/yttria beads and a nucleic acid primer, wherein said primer is complementary to a sequence from a tick-borne pathogen.

14. The kit of claim 13, comprising two nucleic acid primers complementary to a sequence from a tick-borne pathogen.

15. The kit of claim 14, wherein said primers are complementary to a nucleic acid region conserved among multiple different tick-borne pathogens.

16. The kit of claim 15, wherein said multiple different tick-borne pathogens comprise different species of tick-borne pathogens.

17. The kit of claim 15, wherein said multiple different tick-borne pathogens comprise different genus of tick-borne pathogens.

18. The kit of claim 15, wherein said multiple different tick-borne pathogens comprise one or more tick-borne viruses.

19. The kit of claim 14, wherein said primers are complementary to a nucleic acid region flanking a variable region.

20. The kit of claim 19, wherein said variable region comprises different sequences among different tick-borne pathogens.

21. The kit of claim 20, wherein said different tick-borne pathogens comprise different species of tick-borne pathogens.

22. The kit of claim 20, wherein said different tick-borne pathogens comprise different genus of tick-borne pathogens.

23. The kit of claim 20, wherein said different tick-borne pathogens comprise one or more tick-borne viruses.

24. The kit of claim 13, further comprising one or more buffers compatible with mass spectrometry analysis.

25. A reaction mixture comprising a reaction vessel containing zirconia/yttria beads and tick-borne pathogen nucleic acid.
